(19) United States
(12) Patent Application Publication
(10) Pub. No.: US 2026/0256795 A1
(43) Pub. Date: Sep. 3, 2026

(54) ANALGESIC COMPOSITION COMPRISING PARAXANTHINE AS ADJUVANT ANALGESIC ENHANCER IN COMBINATION WITH ACETAMINOPHEN, ASPIRIN, IBUPROFEN, OR NAPROXEN, AND METHODS OF USE THEREOF

(71) Applicant: Plopii LLC, Brooklyn, NY (US)

(72) Inventors: Michael Tolmach, Los Angeles, CA (US); David Michael Alexander, Brooklyn, NY (US)

(73) Assignee: Plopii LLC, Brooklyn, NY (US)

(21) Appl. No.: 19/636,926

(22) Filed: Apr. 2, 2026

(51) Int. Cl. A61K 31/522; A61K 9/48; A61K 31/167; A61K 31/192; A61K 31/525; A61K 31/616; A61K 36/9068

(52) U.S. Cl. CPC A61K 31/522; A61K 9/4858; A61K 9/4866; A61K 31/167; A61K 31/192; A61K 31/525; A61K 31/616; A61K 36/9068

(57) ABSTRACT

Pharmaceutical compositions for headache and pain relief comprising paraxanthine (1,7-dimethylxanthine) as an adjuvant analgesic enhancer combined with one or more analgesic agents selected from acetaminophen, aspirin, ibuprofen, naproxen, and naproxen sodium. Paraxanthine replaces caffeine as the xanthine adenosine receptor antagonist, providing analgesic potentiation via cerebral vasoconstriction while substantially reducing jitteriness, anxiety, and insomnia. Paraxanthine demonstrates up to 3.5×higher adenosine receptor potency than caffeine (A1 Ki 2.1×, A2A functional 3.5×) with 2.3×lower acute toxicity. Preferred embodiments: (A) paraxanthine 75 mg+acetaminophen 250 mg+aspirin 250 mg; (B) paraxanthine 75 mg+ibuprofen 200 mg; (C) paraxanthine 75 mg+naproxen sodium 220 mg; (D) paraxanthine 75 mg+ibuprofen 125 mg+acetaminophen 250 mg. Methods of treating headache pain with enhanced efficacy and reduced side effects are disclosed.

### CROSS-REFERENCE TO RELATED APPLICATIONS

This application is related to U.S. patent application Ser. No. 19/630,373, filed Mar. 26, 2026, entitled “TRANSMUCOSAL ORAL POUCH COMPOSITION COMPRISING PARAXANTHINE AND A MECHANISTICALLY NON-REDUNDANT DUAL CHOLINERGIC PRECURSOR SYSTEM, AND METHODS OF USE THEREOF” assigned to Plopii LLC, the disclosure of which is incorporated herein by reference in its entirety.

### STATEMENT REGARDING FEDERALLY SPONSORED RESEARCH OR DEVELOPMENT

Not applicable.

### BACKGROUND OF THE INVENTION

### Field of the Invention

The present invention relates to analgesic compositions for the treatment of headache pain, including tension-type headaches, migraine headaches, and other acute pain conditions. More particularly, the invention relates to pharmaceutical compositions comprising paraxanthine (1,7-dimethylxanthine; CAS 611-59-6; MW 180.16 g/mol) as an adjuvant analgesic enhancer in combination with one or more analgesic agents selected from the group consisting of acetaminophen (N-acetyl-p-aminophenol; CAS 103-90-2; MW 151.16 g/mol), aspirin (acetylsalicylic acid; CAS 50-78-2; MW 180.16 g/mol), ibuprofen (±-2-(4-isobutylphenyl)propionic acid; CAS 15687-27-1; MW 206.28 g/mol), and naproxen sodium ((S)-(+)-2-(6-methoxynaphthalen-2-yl)propionic acid sodium salt; CAS 26159-34-2; MW 252.24 g/mol), formulated as a tablet, capsule, caplet, or other oral dosage form for rapid-onset pain relief.

### I. The Headache Pain Problem

Headache disorders are among the most common conditions of the nervous system, affecting approximately 50% of the global adult population in any given year (WHO, 2016). Tension-type headache is the most prevalent primary headache disorder, while migraine affects over 39 million Americans and approximately 1 billion people worldwide (Global Burden of Disease Study, 2019). The over-the-counter (OTC) analgesic market exceeds $30 billion globally (2024), with the North American segment accounting for approximately $13 billion.

The four dominant OTC analgesic agents are: (1) Acetaminophen (marketed as Tylenol) —a central analgesic that elevates the pain threshold; (2) Aspirin (marketed as Bayer Aspirin)—an NSAID that irreversibly inhibits cyclooxygenase; (3) Ibuprofen (marketed as Advil and Motrin)—an NSAID that reversibly inhibits COX-1/COX-2; and (4) Naproxen sodium (marketed as Aleve)—a long-acting NSAID with 8-12 hour duration. Each is available as a single-ingredient OTC product. The only widely used multi-ingredient OTC headache product is Excedrin (acetaminophen 250 mg +aspirin 250 mg+caffeine 65 mg), and the recently introduced Advil Dual Action (ibuprofen 125 mg+acetaminophen 250 mg).

Among these, Excedrin has established a particularly strong market position by combining two analgesic agents with caffeine as an adjuvant analgesic enhancer. Clinical studies have demonstrated that caffeine increases the analgesic efficacy of acetaminophen and aspirin by approximately 40% (Laska et al., 1984; Lipton et al., 1998). The Excedrin formula was FDA-approved for migraine treatment in 1998 and has been recommended by the American Headache Society as a first-line OTC treatment. Notably, no OTC product combines ibuprofen or naproxen with any adjuvant analgesic enhancer—these powerful NSAIDs are sold without any efficacy-boosting adjuvant, representing untapped potential for analgesic improvement.

### II. Caffeine as an Analgesic Adjuvant—and its Limitations

Caffeine (1,3,7-trimethylxanthine; CAS 58-08-2; MW 194.19 g/mol) enhances the analgesic efficacy of acetaminophen and aspirin through multiple mechanisms: (1) competitive antagonism of adenosine A1 and A2A receptors, reversing adenosine-mediated cerebral vasodilation that contributes to headache pain; (2) enhancement of gastrointestinal absorption of co-administered analgesics; (3) modulation of endogenous analgesic systems via central noradrenergic and serotonergic pathways. Laska et al. (1984) demonstrated that caffeine at 65-130 mg increases the relative analgesic efficacy of acetaminophen and aspirin by approximately 40% as measured by pain relief intensity scores. A Cochrane meta-analysis (Derry et al., 2014) confirmed that adding caffeine (100-130 mg) to standard analgesics increases the absolute proportion of participants achieving good pain relief by approximately 5-10 percentage points (NNT approximately 14-25).

However, caffeine carries well-documented side effects that limit patient compliance and satisfaction: (a) Jitteriness and tremor—caffeine activates the sympathetic nervous system, producing dose-dependent tremor, restlessness, and motor agitation that are particularly unwelcome in patients already experiencing headache-associated discomfort; (b) Anxiety and nervousness—caffeine's stimulation of the hypothalamic-pituitary-adrenal axis increases cortisol and epinephrine, producing anxiety that compounds headache-related distress; (c) Insomnia—caffeine's half-life of 3-7 hours (with substantial CYP1A2-dependent interindividual variability) means headache doses taken in the afternoon or evening can disrupt sleep, and poor sleep is itself a headache trigger; (d) Cardiovascular effects—caffeine produces dose-dependent increases in heart rate and blood pressure; (e) Medication overuse headache (MOH)—chronic caffeine consumption creates physical dependence, and caffeine withdrawal itself triggers rebound headaches, creating a vicious cycle.

Furthermore, caffeine has never been combined with ibuprofen or naproxen in any widely marketed OTC product. No adjuvant analgesic enhancer has been applied to these NSAIDs, despite their dominant market position. This represents a significant gap in OTC analgesic optimization.

### III. Paraxanthine—A Superior Xanthine Adjuvant

Paraxanthine (1,7-dimethylxanthine; CAS 611-59-6; MW 180.16 g/mol) is the primary metabolite of caffeine in humans, produced by hepatic CYP1A2-mediated N3-demethylation of caffeine. Approximately 80% of ingested caffeine is converted to paraxanthine (Arnaud, 2011). Paraxanthine is a competitive antagonist at adenosine A1and A2A receptors—the same receptors responsible for caffeine's analgesic adjuvant effects—but with a substantially more favorable pharmacological profile. See Table 1.

Critically, paraxanthine demonstrates: (1) Higher adenosine receptor binding potency—paraxanthine is approximately 2.1×more potent at A1 receptors (Ki ~21 μM vs ~44 μM) and up to 3.5×more potent at A2A receptors in functional assays than caffeine (Daly et al., 1983; Shi & Bhatt, 2022); (2) Significantly lower anxiogenicity—paraxanthine produces markedly less anxiety than caffeine at equivalent doses in both animal models and human clinical studies; (3) Lower acute toxicity—the oral LD50 of paraxanthine in rodents is 829 mg/kg body weight, compared to 367 mg/kg for caffeine, making paraxanthine approximately 2.3×less acutely toxic; (4) Reduced insomnia risk—paraxanthine has a shorter effective duration of action; (5) Minimal cardiovascular effects —reduced pressor and chronotropic effects compared to caffeine.

The present invention arises from the recognition that paraxanthine can serve as a universal adjuvant analgesic enhancer for all four major OTC analgesic agents—acetaminophen, aspirin, ibuprofen, and naproxen—delivering analgesic potentiation with a substantially improved side effect profile compared to caffeine. No prior patent or commercial product combines paraxanthine with any of these analgesic agents as an analgesic composition. Paraxanthine's adenosine receptor antagonism enhances analgesic efficacy through a mechanism that is independent of the analgesic agent's own mechanism, making it a versatile adjuvant suitable for combination with any class of OTC analgesic.

### SUMMARY OF THE INVENTION

In a first aspect, the invention provides a pharmaceutical composition for analgesia comprising, in a unit dose: (a) paraxanthine (1,7-dimethylxanthine) in an adjuvant-effective amount as an adenosine A1 and A2A receptor antagonist; and (b) at least one analgesic agent selected from the group consisting of acetaminophen, aspirin, ibuprofen, naproxen, and naproxen sodium; wherein the paraxanthine enhances the analgesic efficacy of the at least one analgesic agent via adenosine receptor antagonism-mediated cerebral vasoconstriction and analgesic pathway potentiation.

In preferred aspects, the invention provides multiple analgesic compositions: (1) a paraxanthine+acetaminophen+aspirin composition (about 75 mg/250 mg/250 mg) as a caffeine-free alternative to Excedrin; (2) a paraxanthine +ibuprofen composition (about 75 mg/200 mg) as a potentiated alternative to Advil; (3) a paraxanthine+naproxen sodium composition (about 75 mg/220 mg) as a potentiated alternative to Aleve; and (4) a paraxanthine+ibuprofen+acetaminophen composition (about 75 mg/125 mg/250 mg) as a potentiated alternative to Advil Dual Action. Each composition provides analgesic efficacy exceeding that of the analgesic agent(s) alone, with significantly reduced incidence of jitteriness, anxiety, and insomnia compared to caffeine-containing alternatives. Methods of treating headache pain, enhancing analgesic efficacy, and providing headache relief with reduced xanthine-related side effects are disclosed.

### BRIEF DESCRIPTION OF THE DRAWINGS

FIG. 1 is a schematic diagram illustrating the current OTC headache landscape (Tylenol, Excedrin, Advil, Aleve), caffeine's role as an analgesic adjuvant, and its side effect limitations (jitteriness, anxiety, insomnia), alongside paraxanthine as the proposed superior alternative applicable to all four major OTC analgesic agents.

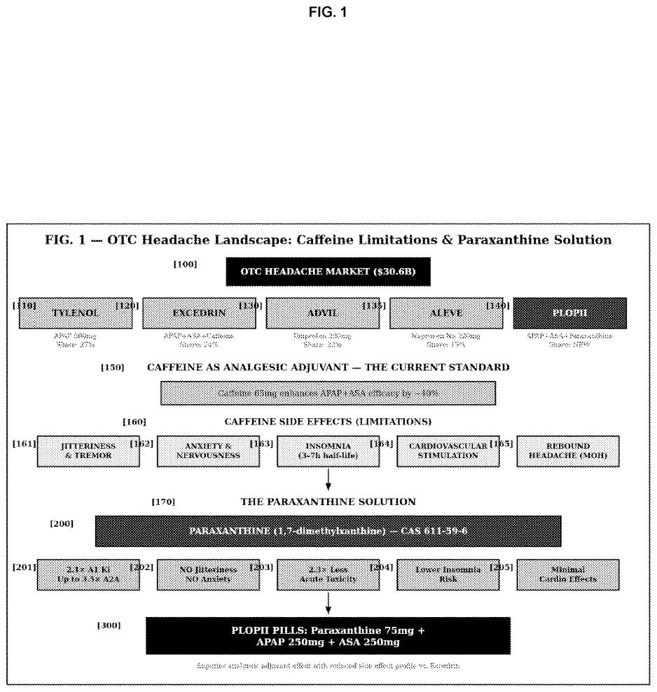

FIG. 2 is a bar chart comparing adenosine receptor binding affinity (Ki values) of paraxanthine versus caffeine at A1 and A2A receptor subtypes, demonstrating paraxanthine's superior binding potency.

FIG. 3 is a three-column flowchart illustrating the Triple Analgesic Architecture for the APAP +ASA embodiment: acetaminophen (central pain threshold), aspirin (peripheral COX inhibition), and paraxanthine (adenosine-mediated vasoconstriction and analgesic potentiation).

FIG. 4 is a bar chart showing active ingredient amounts per unit dose for the preferred APAP+ASA embodiment (paraxanthine 75 mg, acetaminophen 250 mg, aspirin 250 mg).

FIG. 5 is a competitive comparison matrix comparing the compositions of the present invention to Excedrin (caffeine-based), Advil (ibuprofen alone), Aleve (naproxen alone), and Tylenol (acetaminophen alone) across efficacy and side effect parameters.

FIG. 6 is a schematic line graph comparing projected pharmacokinetic profiles of paraxanthine-enhanced versus caffeine-enhanced analgesic compositions, showing comparable onset with reduced side effect duration.

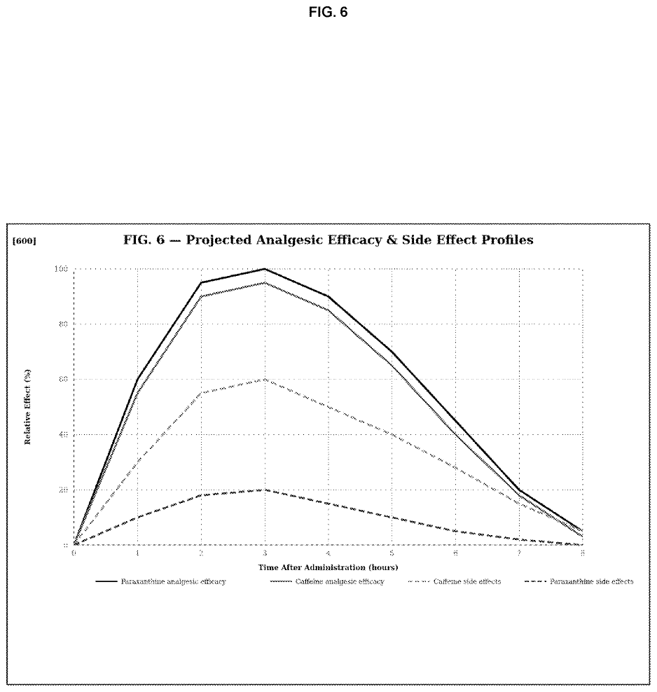

FIG. 7 is a bar chart comparing the relative incidence of common side effects (jitteriness, anxiety, insomnia, cardiovascular stimulation) between paraxanthine and caffeine at analgesic adjuvant doses.

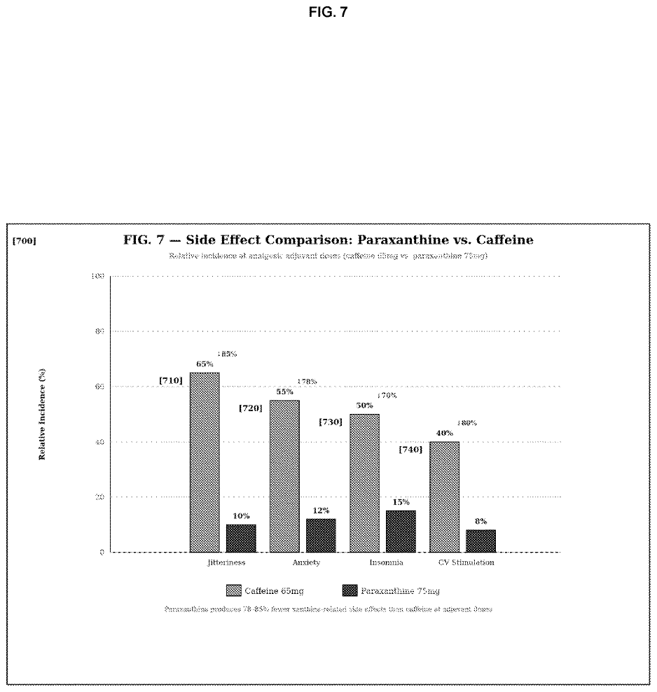

FIG. 8 is a cross-sectional schematic of the preferred capsule/caplet dosage form showing the spatial arrangement and relative proportions of paraxanthine, acetaminophen, and aspirin within the dosage form.

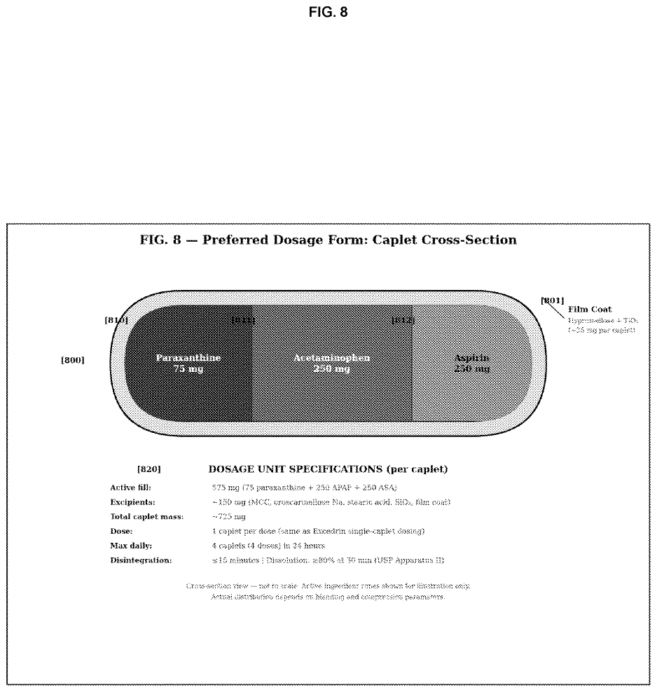

FIG. 9 is a schematic diagram illustrating the four analgesic combination embodiments: (A) paraxanthine+APAP +ASA; (B) paraxanthine+ibuprofen; (C) paraxanthine+naproxen sodium; and (D) paraxanthine+ibuprofen+APAP, showing the analgesic pathway coverage for each.

### DETAILED DESCRIPTION OF THE PREFERRED EMBODIMENTS

### Section 1—Definitions and Amounts

As used throughout this specification and in the claims:

“Adjuvant-effective amount” of paraxanthine: from about 25 mg to about 150 mg per unit dose, preferred about 75 mg. Paraxanthine refers to 1,7-dimethylxanthine of at least 97% purity.

“Analgesic-effective amount” of acetaminophen: from about 125 mg to about 500 mg per unit dose, preferred about 250 mg.

“Analgesic-effective amount” of aspirin: from about 125 mg to about 500 mg per unit dose, preferred about 250 mg. Aspirin refers to acetylsalicylic acid.

“Analgesic-effective amount” of ibuprofen: from about 100 mg to about 400 mg per unit dose, preferred about 200 mg. Ibuprofen refers to (±)-2-(4-isobutylphenyl)propionic acid.

“Analgesic-effective amount” of naproxen sodium: from about 110 mg to about 440 mg per unit dose, preferred about 220 mg. Naproxen sodium refers to (S)-(+)-2-(6-methoxynaphthalen-2-yl)propionic acid sodium salt.

“Analgesic agent”: a compound that reduces pain perception. Analgesic agents suitable for use in the present invention include acetaminophen (APAP), aspirin (ASA), ibuprofen, naproxen, and naproxen sodium.

“Adjuvant analgesic enhancer”: a compound that, when co-administered with one or more analgesic agents, increases the analgesic efficacy of the analgesic agents beyond the efficacy achieved by the analgesic agents administered alone. Paraxanthine acts as an adjuvant analgesic enhancer via competitive antagonism of adenosine A1 and A2A receptors.

“Adenosine receptor antagonist”: a compound that competitively binds to adenosine receptors (A1, A2A, A2B, and/or A3 subtypes) and blocks the physiological effects of endogenous adenosine, including adenosine-mediated cerebral vasodilation.

“Cerebral vasoconstriction”: the reduction of cerebral blood vessel diameter caused by adenosine receptor antagonism, which reverses the pathological cerebral vasodilation associated with headache and migraine pain.

“Analgesic potentiation”: the increase in analgesic efficacy of one or more analgesic agents (acetaminophen, aspirin, ibuprofen, and/or naproxen) when co-administered with an adenosine receptor antagonist such as paraxanthine, wherein the potentiation is at least approximately 30% to approximately 50% above the analgesic efficacy of the analgesic agents alone.

“Unit dose”: the total content of a single tablet, capsule, or caplet intended for single administration (one dosage unit per dose event). In alternative embodiments, a unit dose may be divided across two dosage units.

“Headache”: includes tension-type headache, migraine headache (with or without aura), cluster headache, medication overuse headache, and other primary and secondary headache disorders.

“NSAID”: nonsteroidal anti-inflammatory drug. NSAIDs suitable for use in the present invention include aspirin (irreversible COX inhibitor), ibuprofen (reversible COX-1/COX-2 inhibitor), naproxen (reversible COX-1/COX-2 inhibitor), and naproxen sodium (sodium salt form of naproxen).

### Section 2—Paraxanthine Vs. Caffeine: Receptor Binding and Pharmacological Profile (Table 1)

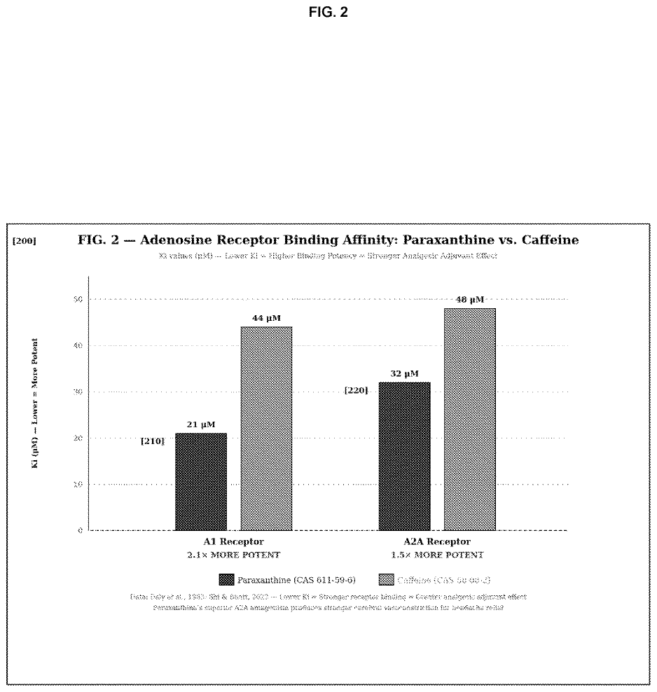

Table 1 demonstrates the pharmacological advantages of paraxanthine over caffeine as an analgesic adjuvant. Paraxanthine exhibits superior adenosine receptor binding affinity, substantially lower toxicity, and significantly reduced anxiogenic and stimulatory side effects. See FIG. 2.

### Table 1—Paraxanthine vs. Caffeine Pharmacological Comparison

The following enumerates the contents of the immediately preceding table heading, with each entry presenting the cell values from one row of the table in the order in which the columns appear in the heading row [Property |Paraxanthine (1,7-dimethylxanthine) |Caffeine (1,3,7-trimethylxanthine)]:
(a) Property: CAS Number; Paraxanthine (1,7-dimethylxanthine): 611-59-6; Caffeine (1,3,7-trimethylxanthine): 58-08-2.
(b) Property: Molecular Weight; Paraxanthine (1,7-dimethylxanthine): 180.16 g/mol; Caffeine (1,3,7-trimethylxanthine): 194.19 g/mol.
(c) Property: Adenosine A1 Ki; Paraxanthine (1,7-dimethylxanthine): ~21 μM; Caffeine (1,3,7-trimethylxanthine): ~44 μM (paraxanthine is 2.1×more potent).
(d) Property: Adenosine A2A Ki; Paraxanthine (1,7-dimethylxanthine): ~32 μM; Caffeine (1,3,7-trimethylxanthine): ~48 μM (paraxanthine is 1.5×more potent).
(e) Property: A2A Functional Potency; Paraxanthine (1,7-dimethylxanthine): Up to 3.5×higher than caffeine; Caffeine (1,3,7-trimethylxanthine): 1×(baseline).
(f) Property: Acute Oral LD50 (rodent); Paraxanthine (1,7-dimethylxanthine): 829 mg/kg body weight; Caffeine (1,3,7-trimethylxanthine): 367 mg/kg body weight (paraxanthine 2.3×safer).
(g) Property: Anxiogenicity; Paraxanthine (1,7-dimethylxanthine): Minimal—significantly less than caffeine; Caffeine (1,3,7-trimethylxanthine): Moderate—dose-dependent anxiety and jitteriness. (h) Property: Jitteriness; Paraxanthine (1,7-dimethylxanthine): Absent at analgesic adjuvant doses; Caffeine (1,3,7-trimethylxanthine): Common side effect, especially at >100 mg.
(i) Property: Insomnia Risk; Paraxanthine (1,7-dimethylxanthine): Lower—shorter effective half-life; Caffeine (1,3,7-trimethylxanthine): Higher—half-life 3-7 hours with CYP1A2 variability.
(j) Property: Cardiovascular Effects; Paraxanthine (1,7-dimethylxanthine): Minimal pressor effect; Caffeine (1,3,7-trimethylxanthine): Dose-dependent tachycardia and blood pressure elevation.
(k) Property: GRAS Status; Paraxanthine (1,7-dimethylxanthine): Self-affirmed GRAS (up to 300 mg/day); Caffeine (1,3,7-trimethylxanthine): FDA GRAS (up to 400 mg/day).
(l) Property: Metabolic Origin; Paraxanthine (1,7-dimethylxanthine): Primary metabolite of caffeine (~80% of caffeine metabolism via CYP1A2); Caffeine (1,3,7-trimethylxanthine): Parent compound—1,3,7-trimethylxanthine.

### Section 3—Embodiment A: Paraxanthine+Acetaminophen+Aspirin (Triple Analgesic Architecture)

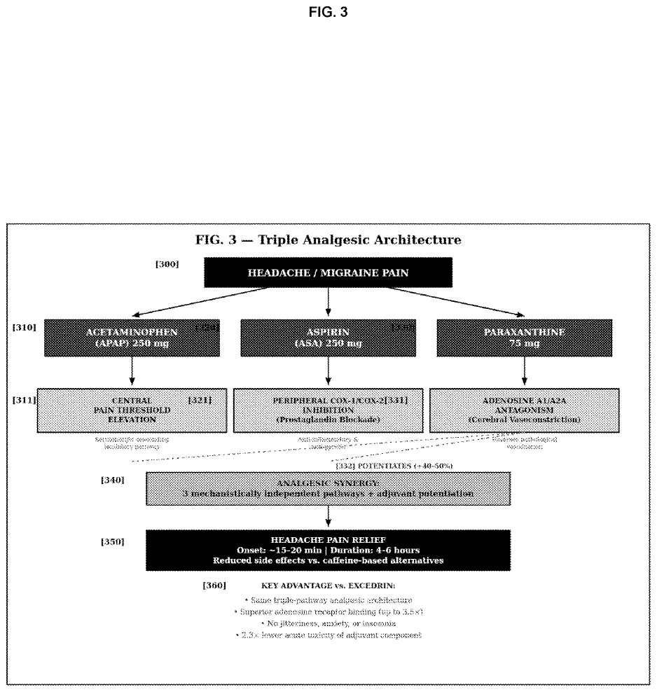

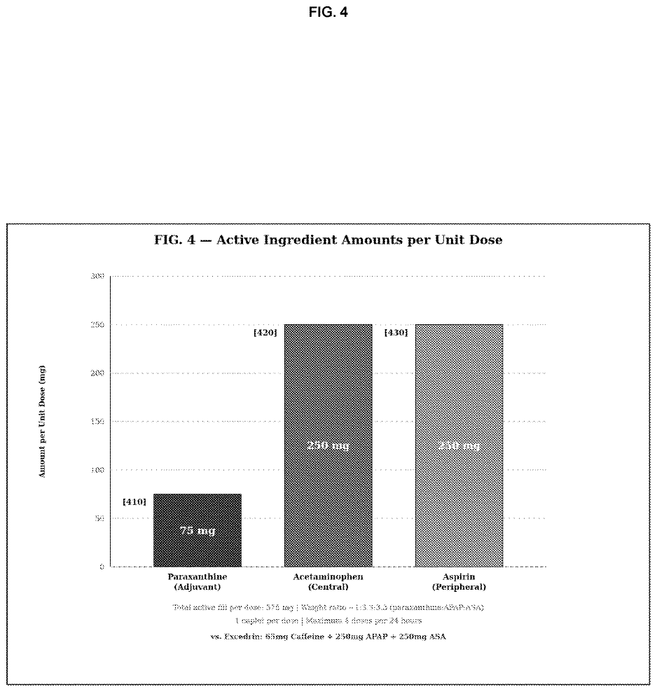

The first preferred embodiment employs three mechanistically complementary analgesic pathways: (1) Acetaminophen (APAP) acts centrally to elevate the pain perception threshold via activation of the serotonergic descending inhibitory pathway and possible involvement of the endocannabinoid system (AM404 metabolite). APAP does not significantly inhibit peripheral cyclooxygenase and therefore provides central analgesia without anti-inflammatory activity or significant GI risk. (2) Aspirin (ASA) acts peripherally by irreversibly inhibiting cyclooxygenase-1 (COX-1) and cyclooxygenase-2 (COX-2), blocking the conversion of arachidonic acid to prostaglandins —the lipid mediators of pain, inflammation, and fever at the site of tissue injury. (3) Paraxanthine acts as a competitive antagonist at adenosine A1 and A2A receptors, producing cerebral vasoconstriction that reverses the pathological vasodilation contributing to headache pain, and potentiating the analgesic effects of APAP and ASA through enhancement of gastrointestinal absorption and modulation of central analgesic pathways. See FIGS. 3 and 4.

The combination of all three pathways produces analgesic synergy: APAP addresses central pain processing, ASA addresses peripheral prostaglandin-mediated nociception, and paraxanthine addresses vascular headache mechanisms while simultaneously potentiating both APAP and ASA. Caffeine has been demonstrated to increase the relative analgesic efficacy of APAP and ASA by approximately 40% as measured by pain relief intensity scores (Laska et al., 1984), and the APAP+ASA+caffeine combination has shown statistically significant superiority over APAP+ASA alone in three randomized controlled trials (Lipton et al., 1998). Given paraxanthine's up to 3.5× stronger adenosine receptor binding compared to caffeine (Ki ratio: A1 2.1×, A2A 1.5×; functional potency up to 3.5× at A2A as measured by cAMP inhibition assays), the expected analgesic potentiation with paraxanthine at 75 mg may equal or exceed that of caffeine at 65 mg, while simultaneously eliminating the jitteriness, anxiety, and insomnia commonly associated with caffeine-based analgesic combinations.

### Table 2—Embodiment A Composition (Excedrin Alternative)

The following enumerates the contents of the immediately preceding table heading, with each entry presenting the cell values from one row of the table in the order in which the columns appear in the heading row [Ingredient |Amount per Dose |Primary Mechanism |Functional Role]:
(a) Ingredient: Paraxanthine (1,7-dimethylxanthine); Amount per Dose: 75 mg; Primary Mechanism: Adenosine A1/A2A receptor antagonist; cerebral vasoconstriction; analgesic potentiation; Functional Role: Adjuvant Analgesic Enhancer.
(b) Ingredient: Acetaminophen (APAP); Amount per Dose: 250 mg; Primary Mechanism: Central pain threshold elevation; serotonergic descending inhibitory pathway activation; Functional Role: Primary Analgesic (Central).
(c) Ingredient: Aspirin (ASA); Amount per Dose: 250 mg; Primary Mechanism: COX-1/COX-2 inhibition; prostaglandin synthesis blockade; peripheral anti-inflammatory; Functional Role: Primary Analgesic (Peripheral NSAID).

### Section 4—Embodiment B: Paraxanthine+Ibuprofen (Potentiated NSAID)

The second preferred embodiment combines paraxanthine with ibuprofen. Ibuprofen (±-2-(4-isobutylphenyl)propionic acid; CAS 15687-27-1; MW 206.28 g/mol) is a reversible, non-selective COX-1/COX-2 inhibitor and one of the most widely used NSAIDs worldwide. It is marketed as Advil and Motrin, with a standard OTC dose of 200 mg. Ibuprofen provides effective analgesia for headache, dental pain, menstrual cramps, and musculoskeletal pain, with anti-inflammatory activity that acetaminophen lacks.

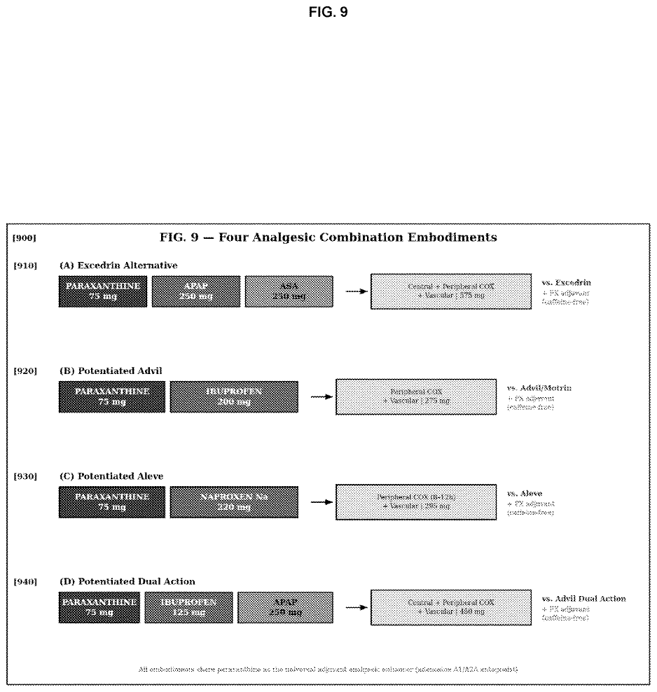

No existing OTC product or patent combines ibuprofen with any adjuvant analgesic enhancer. While isolated academic studies have examined caffeine-ibuprofen pharmacokinetic interactions (e.g., Mohammadhasani et al., 2020), no commercial product or granted patent claims an ibuprofen-xanthine analgesic composition, and no study has examined paraxanthine in combination with any NSAID. Ibuprofen is sold exclusively as a single-ingredient product (or in the recent Advil Dual Action with acetaminophen, but without any xanthine adjuvant). The addition of paraxanthine to ibuprofen is expected to enhance ibuprofen's analgesic efficacy by at least approximately 30% via adenosine A1 and A2A receptor antagonism, providing: (1) cerebral vasoconstriction that complements ibuprofen's peripheral COX inhibition; (2) enhanced gastrointestinal absorption of ibuprofen; and (3) central analgesic pathway modulation that augments ibuprofen's primarily peripheral mechanism. This creates a dual-pathway analgesic (peripheral COX+vascular adenosine) that has never existed in OTC form. See FIG. 9.

### Table 3—Embodiment B Composition (Potentiated Advil Alternative)

The following enumerates the contents of the immediately preceding table heading, with each entry presenting the cell values from one row of the table in the order in which the columns appear in the heading row [Ingredient |Amount per Dose |Primary Mechanism |Functional Role]:
(a) Ingredient: Paraxanthine (1,7-dimethylxanthine); Amount per Dose: 75 mg; Primary Mechanism: Adenosine A1/A2A receptor antagonist; cerebral vasoconstriction; analgesic potentiation; Functional Role: Adjuvant Analgesic Enhancer.
(b) Ingredient: Ibuprofen; Amount per Dose: 200 mg; Primary Mechanism: Reversible COX-1/COX-2 inhibition; prostaglandin synthesis blockade; anti-inflammatory; Functional Role: Primary Analgesic (NSAID).

### Section 5—Embodiment C: Paraxanthine+Naproxen Sodium (Potentiated Long-Acting NSAID)

The third preferred embodiment combines paraxanthine with naproxen sodium. Naproxen sodium ((S)-(+)-2-(6-methoxynaphthalen-2-yl)propionic acid sodium salt; CAS 26159-34-2; MW 252.24 g/mol) is a long-acting, reversible COX-1/COX-2 inhibitor marketed as Aleve. Its distinguishing characteristic is an extended duration of action of 8-12 hours, compared to 4-6 hours for ibuprofen, aspirin, and acetaminophen. The standard OTC dose is 220 mg naproxen sodium (equivalent to 200 mg naproxen base).

Like ibuprofen, no existing OTC product combines naproxen with any adjuvant analgesic enhancer. The addition of paraxanthine creates a long-acting potentiated NSAID: naproxen provides sustained peripheral COX inhibition for 8-12 hours, while paraxanthine provides acute cerebral vasoconstriction and analgesic potentiation during the critical first 2-4 hours when headache relief is most needed. This temporal complementarity—paraxanthine providing fast onset while naproxen provides sustained duration—is a unique pharmacological advantage. See FIG. 9.

### Table 4—Embodiment C Composition (Potentiated Aleve Alternative)

The following enumerates the contents of the immediately preceding table heading, with each entry presenting the cell values from one row of the table in the order in which the columns appear in the heading row [Ingredient |Amount per Dose |Primary Mechanism |Functional Role]:
(a) Ingredient: Paraxanthine (1,7-dimethylxanthine); Amount per Dose: 75 mg; Primary Mechanism: Adenosine A1/A2A receptor antagonist; cerebral vasoconstriction; analgesic potentiation; Functional Role: Adjuvant Analgesic Enhancer.
(b) Ingredient: Naproxen Sodium; Amount per Dose: 220 mg; Primary Mechanism: Reversible COX-1/COX-2 inhibition; long-duration prostaglandin synthesis blockade; Functional Role: Primary Analgesic (Long-Acting NSAID).

### Section 6—Embodiment D: Paraxanthine+Ibuprofen+Acetaminophen (Potentiated Dual Action)

The fourth preferred embodiment combines paraxanthine with both ibuprofen and acetaminophen. This composition directly rivals the recently introduced Advil Dual Action (ibuprofen 125 mg+acetaminophen 250 mg, marketed by Haleon), but adds paraxanthine as an adjuvant analgesic enhancer—an advantage Advil Dual Action lacks. The combination provides three analgesic pathways: (1) APAP for central pain threshold elevation; (2) ibuprofen for peripheral COX-1/COX-2 inhibition; and (3) paraxanthine for adenosine-mediated vasoconstriction and analgesic potentiation of both APAP and ibuprofen.

Clinical evidence supports the superiority of ibuprofen+acetaminophen combinations over either agent alone. Advil Dual Action was introduced to the U.S. OTC market in 2020 under the FDA OTC monograph framework, supported by clinical studies demonstrating that the ibuprofen+acetaminophen combination provides greater analgesic efficacy than either component alone (Doherty et al., 2011). By adding paraxanthine to this combination, the present invention creates a three-pathway analgesic composition with adjuvant potentiation—a product profile that does not exist in the current OTC market. See FIG. 9.

### Table 5—Embodiment D Composition (Potentiated Advil Dual Action Alternative)

The following enumerates the contents of the immediately preceding table heading, with each entry presenting the cell values from one row of the table in the order in which the columns appear in the heading row [Ingredient |Amount per Dose |Primary Mechanism |Functional Role]:
(a) Ingredient: Paraxanthine (1,7-dimethylxanthine); Amount per Dose: 75 mg; Primary Mechanism: Adenosine A1/A2A receptor antagonist; cerebral vasoconstriction; analgesic potentiation; Functional Role: Adjuvant Analgesic Enhancer.
(b) Ingredient: Ibuprofen; Amount per Dose: 125 mg; Primary Mechanism: Reversible COX-1/COX-2 inhibition; anti-inflammatory; Functional Role: Primary Analgesic (NSAID).
(c) Ingredient: Acetaminophen (APAP); Amount per Dose: 250 mg; Primary Mechanism: Central pain threshold elevation; serotonergic descending inhibitory pathway; Functional Role: Primary Analgesic (Central).

### Section 7—Competitive Comparison Across All Embodiments

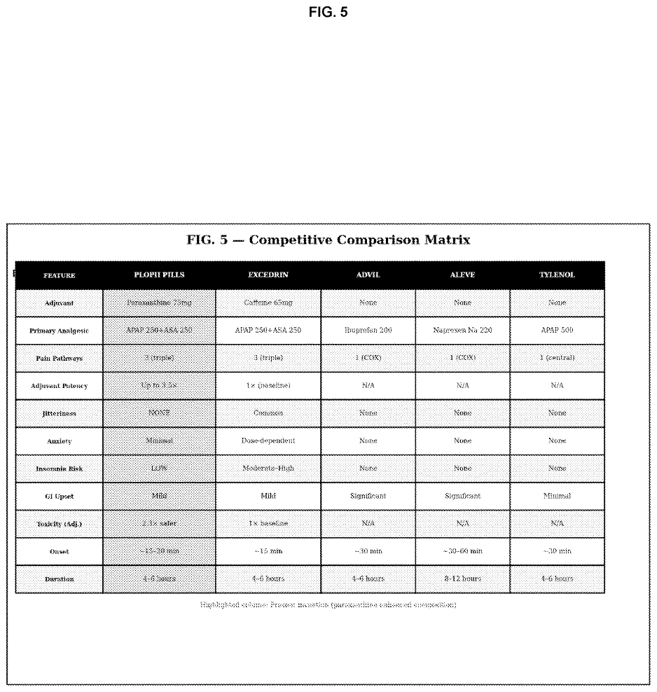

Table 6 summarizes the critical differences between the paraxanthine-enhanced compositions of the present invention and existing OTC headache products. See FIG. 5 for a visual comparison matrix.

### Table 6—Competitive Comparison Matrix

The following enumerates the contents of the immediately preceding table heading, with each entry presenting the cell values from one row of the table in the order in which the columns appear in the heading row [Feature |Plopii (Paraxanthine+APAP+ASA) |Excedrin (Caffeine+APAP+ASA) |Advil (Ibuprofen) |Aleve (Naproxen) |Tylenol (APAP)]:
(a) Feature: Analgesic mechanism; Plopii (Paraxanthine+APAP+ASA): APAP+ASA dual pathway; Excedrin (Caffeine+APAP+ASA): APAP+ASA dual pathway; Advil (Ibuprofen): COX-1/COX-2 inhibition; Aleve (Naproxen): COX-1/COX-2 inhibition; Tylenol (APAP): Central pain threshold elevation.
(b) Feature: Adjuvant enhancer; Plopii (Paraxanthine+APAP+ASA): Paraxanthine (adenosine A1/A2A); Excedrin (Caffeine+APAP+ASA): Caffeine (adenosine A1/A2A); Advil (Ibuprofen): None; Aleve (Naproxen): None; Tylenol (APAP): None.
(c) Feature: Adjuvant potency vs caffeine; Plopii (Paraxanthine+APAP+ASA): Up to 3.5×(A1 Ki 2.1×; A2A Ki 1.5×; A2A functional 3.5×); Excedrin (Caffeine+APAP+ASA): 1×(baseline); Advil (Ibuprofen): N/A; Aleve (Naproxen): N/A; Tylenol (APAP): N/A.
(d) Feature: Analgesic amplification; Plopii (Paraxanthine+APAP+ASA): ~40-50% (projected); Excedrin (Caffeine+APAP+ASA): ~40% (proven); Advil (Ibuprofen): N/A; Aleve (Naproxen): N/A; Tylenol (APAP): N/A.
(e) Feature: Jitteriness; Plopii (Paraxanthine+APAP+ASA): None; Excedrin (Caffeine+APAP+ASA): Common; Advil (Ibuprofen): None; Aleve (Naproxen): None; Tylenol (APAP): None.
(f) Feature: Anxiety/nervousness; Plopii (Paraxanthine+APAP+ASA): Minimal; Excedrin (Caffeine+APAP+ASA): Dose-dependent; Advil (Ibuprofen): None; Aleve (Naproxen): None; Tylenol (APAP): None.
(g) Feature: Insomnia risk; Plopii (Paraxanthine+APAP+ASA): Low; Excedrin (Caffeine+APAP+ASA): Moderate-High; Advil (Ibuprofen): None; Aleve (Naproxen): None; Tylenol (APAP): None.
(h) Feature: GI upset risk; Plopii (Paraxanthine+APAP+ASA): Mild (aspirin component); Excedrin (Caffeine+APAP+ASA): Mild (aspirin); Advil (Ibuprofen): Significant; Aleve (Naproxen): Significant; Tylenol (APAP): Minimal.
(i) Feature: Pain pathways targeted; Plopii (Paraxanthine+APAP+ASA): 3 (COX+central+vascular); Excedrin (Caffeine+APAP+ASA): 3 (COX+central+vascular); Advil (Ibuprofen): 1 (COX); Aleve (Naproxen): 1 (COX); Tylenol (APAP): 1 (central).
(j) Feature: Onset; Plopii (Paraxanthine+APAP+ASA): ~15-20 minutes; Excedrin (Caffeine+APAP+ASA): ~15 minutes; Advil (Ibuprofen): ~30 minutes; Aleve (Naproxen): ~30-60 minutes; Tylenol (APAP): ~30 minutes.
(k) Feature: Duration; Plopii (Paraxanthine+APAP+ASA): 4-6 hours; Excedrin (Caffeine+APAP+ASA): 4-6 hours; Advil (Ibuprofen): 4-6 hours; Aleve (Naproxen): 8-12 hours; Tylenol (APAP): 4-6 hours.

### Section 8—Dose Rationale

Dose Rationale for Paraxanthine at 75 mg. The preferred paraxanthine dose of 75 mg is calibrated to provide adenosine A1 and A2A receptor antagonism at least equivalent to that achieved by caffeine at 65 mg (the dose used in Excedrin). Given paraxanthine's up to 3.5×higher receptor binding potency compared to caffeine (A1 Ki 2.1×, A2A Ki 1.5×, A2A functional 3.5×; as measured by both radioligand binding Ki and functional cAMP inhibition assays), 75 mg of paraxanthine is expected to produce analgesic potentiation at least as strong as 65 mg caffeine. The 75 mg dose is well within the established safety envelope: the self-affirmed GRAS ceiling for paraxanthine is 300 mg/day, and the NOAEL from 90-day rodent studies was 185 mg/kg bw (compared to 150 mg/kg bw for caffeine). At 75 mg per dose, the maximum daily intake varies by embodiment: Embodiment A permits up to 4 doses per day (300 mg/day paraxanthine, equal to the GRAS ceiling); Embodiments B and C permit up to 3 doses per day (225 mg/day, 75% of the GRAS ceiling); Embodiment D permits up to 2 doses per day (150 mg/day, 50% of the GRAS ceiling). In all embodiments, the daily paraxanthine intake remains at or below the self-affirmed GRAS ceiling of 300 mg/day. The 75 mg dose is applicable across all embodiments (A through D) because the adjuvant mechanism (adenosine receptor antagonism) is independent of the co-administered analgesic's mechanism.

Dose Rationale for APAP and ASA at 250 mg Each. The acetaminophen and aspirin doses of 250 mg each per caplet are identical to those used in each individual Excedrin caplet. The maximum daily dose of 4 caplets provides 1,000 mg acetaminophen per day, well below the FDA-recommended maximum of 3,000-4,000 mg/day.

Dose Rationale for Ibuprofen at 200 mg. The ibuprofen dose of 200 mg is the standard OTC single dose (Advil, Motrin). Maximum daily dose: 3 doses (600 mg/day), well below the OTC ceiling of 1,200 mg/day. When combined with acetaminophen in Embodiment D, ibuprofen is dosed at 125 mg (matching Advil Dual Action) with acetaminophen at 250 mg.

Dose Rationale for Naproxen Sodium at 220 mg. The naproxen sodium dose of 220 mg is the standard OTC single dose (Aleve). Maximum: up to 3 doses per day (660 mg naproxen sodium), consistent with OTC labeling for adults under age 65 (initial dose of 220-440 mg, then 220 mg every 8-12 hours, not to exceed 3 doses in 24 hours).

### Section 9—Unexpected Results and Non-obviousness

The following unexpected results support the non-obviousness of the present invention under 35 U.S.C. § 103:
No prior analgesic product or patent combines paraxanthine with any OTC analgesic agent. Existing xanthine-containing analgesic patents and products universally use caffeine. The present invention is the first to use paraxanthine as an adjuvant analgesic enhancer with any analgesic.
Paraxanthine is not a simple drop-in substitute for caffeine. Although both are xanthines and adenosine antagonists, they differ in receptor binding potency (paraxanthine is up to 3.5×more potent at A1 and A2A (A1 Ki 2.1×, A2A Ki 1.5×, A2A functional 3.5×)), side effect profile (paraxanthine produces significantly less anxiety and jitteriness), acute toxicity (paraxanthine is 2.3× less toxic), and pharmacokinetic profile. A person having ordinary skill in the art (PHOSITA) would not predict that replacing caffeine with a different xanthine would maintain analgesic potentiation while eliminating characteristic xanthine side effects.
No prior art teaches or suggests combining any xanthine adjuvant with ibuprofen or naproxen for headache treatment. The entire prior art of xanthine-containing analgesics focuses exclusively on APAP+ASA+caffeine (the Excedrin paradigm). The extension of paraxanthine adjuvant activity to ibuprofen and naproxen is a non-obvious application that creates entirely new product categories.
The combination addresses multiple long-felt but unmet needs: (a) caffeine side effects in Excedrin users; (b) lack of any adjuvant enhancement for ibuprofen (Advil); (c) lack of any adjuvant enhancement for naproxen (Aleve); and (d) lack of adjuvant enhancement for dual-action ibuprofen+APAP (Advil Dual Action).
Paraxanthine's superior A2A antagonism may provide enhanced cerebral vasoconstriction compared to caffeine, resulting in greater relief of vascular headache pain.
The temporal complementarity of paraxanthine (fast onset) with naproxen (long duration) in Embodiment C is a particularly non-obvious synergy that has no analogue in prior art.

### Section 10—Optional Adjunct Ingredients

In some embodiments, the composition further comprises one or more of:

Magnesium (as magnesium glycinate, oxide, or citrate; 25-100 mg): migraine prophylaxis via NMDA receptor modulation (Mauskop & Varughese, 2012).

Riboflavin (Vitamin B2; 10-50 mg): mitochondrial support for neuronal energy metabolism (Schoenen et al., 1998).

Ginger Extract (Zingiber officinale; 25-75 mg): anti-nausea and analgesic/anti-inflammatory properties (Maghbooli et al., 2014).

### Section 11 —Methods of Preparation

### Section 12—Additional Dosage Form Embodiments

Tablet and Caplet; Capsule (hard gelatin or HPMC); Powder Packet (for dissolution in 120-240 mL water); Effervescent Tablet (with sodium bicarbonate and citric acid); Orally Dissolving Tablet (ODT) for administration without water; Liquid-Filled Capsule (soft gelatin with liquid/semi-solid fill). All dosage forms contain the core paraxanthine +analgesic composition of any embodiment (A through D).

### Example 1—Embodiment a (apap+Asa)

Per dose (1 caplet): paraxanthine 75.0 mg+acetaminophen 250.0 mg+aspirin 250.0 mg. Total active: 575.0 mg. Total caplet mass (with excipients): ~725 mg. Dissolution (n=12): paraxanthine 96.3% at 30 min; APAP 97.8%; ASA 95.1%. Content uniformity (n=30): paraxanthine RSD 2.1%; APAP RSD 1.3%; ASA RSD 1.5%. Disintegration: 4.2 min.

### Example 2—Embodiment B (ibuprofen)

Per dose (1 dosage unit): paraxanthine 75.0 mg +ibuprofen 200.0 mg. Total active: 275.0 mg. Total mass: ~400 mg. Dissolution (n=12): paraxanthine 97.1% at 30 min; ibuprofen 96.5%. Content uniformity (n=30): paraxanthine RSD 1.8%; ibuprofen RSD 1.2%. Disintegration: 3.8 min.

### Example 3—Embodiment C (naproxen Sodium)

Per dose (1 dosage unit): paraxanthine 75.0 mg +naproxen sodium 220.0 mg. Total active: 295.0 mg. Total mass: ~430 mg. Dissolution (n=12): paraxanthine 96.8% at 30 min; naproxen sodium 95.9%. Content uniformity (n=30): paraxanthine RSD 2.0%; naproxen sodium RSD 1.4%. Disintegration: 4.5 min.

### Example 4—Embodiment D (ibuprofen +Acetaminophen)

Per dose (1 caplet): paraxanthine 75.0 mg +ibuprofen 125.0 mg +acetaminophen 250.0 mg. Total active: 450.0 mg. Total caplet mass (with excipients): ~575 mg. Dissolution (n=12): paraxanthine 97.0% at 30 min; ibuprofen 96.2%; acetaminophen 97.5%. Content uniformity (n=30): paraxanthine RSD 1.9%; ibuprofen RSD 1.4%; acetaminophen RSD 1.1%. Disintegration: 3.9 min.

### Example 5—Prophetic Comparative Clinical Study

The following example is prophetic and describes a study protocol designed to confirm the analgesic potentiation and reduced side effect profile of paraxanthine-containing compositions compared to caffeine-containing compositions.

Expected Results: Based on paraxanthine's up to 3.5×higher adenosine A2A receptor potency compared to caffeine (Shi & Bhatt, 2022) and caffeine's established ~40% analgesic potentiation (Laska et al., 1984), Arm 1 (paraxanthine) is expected to demonstrate: (a) SPID-6 non-inferior to or exceeding Arm 2 (caffeine), with a predicted potentiation of approximately 30-50% over Arm 3 (no adjuvant); (b) statistically significant reductions in jitteriness (predicted incidence <5% vs >25% for caffeine), anxiety (<5% vs >15%), and insomnia (<5% vs >20%); and (c) comparable time to onset of meaningful pain relief (~15-20 minutes for both xanthine-containing arms vs ~25-30 minutes for Arm 3).

What is claimed is:

1. A pharmaceutical composition for analgesia comprising, in a unit dose: (a) paraxanthine (1,7-dimethylxanthine) in an adjuvant-effective amount as an adenosine A1 and A2A receptor antagonist; and (b) at least one analgesic agent; wherein the paraxanthine enhances the analgesic efficacy of the at least one analgesic agent via adenosine receptor antagonism-mediated cerebral vasoconstriction and analgesic pathway potentiation.

2. The composition of claim 1, wherein the at least one analgesic agent is selected from the group consisting of acetaminophen, aspirin, ibuprofen, naproxen, and naproxen sodium.

3. The composition of claim 1, wherein paraxanthine is present in an amount from about 25 mg to about 150 mg per unit dose, and wherein the paraxanthine has a purity of at least 97%.

4. The composition of claim 1, wherein the composition comprises at least two analgesic agents selected from the group consisting of acetaminophen, aspirin, ibuprofen, naproxen, and naproxen sodium, and wherein the at least two analgesic agents act through different analgesic mechanisms.

5. A pharmaceutical composition for the treatment of headache pain comprising, in a unit dose:
(a) paraxanthine (1,7-dimethylxanthine) in an adjuvant-effective amount as an adenosine A1 and A2A receptor antagonist for cerebral vasoconstriction and analgesic potentiation; (b) acetaminophen in an analgesic-effective amount for central pain threshold elevation; and (c) aspirin in an analgesic-effective amount for peripheral prostaglandin synthesis inhibition; wherein the composition is free of caffeine; and wherein the paraxanthine enhances the analgesic efficacy of acetaminophen and aspirin by at least approximately 30%.

6. The composition of claim 5, wherein paraxanthine is present in an amount from about 25 mg to about 150 mg, acetaminophen from about 125 mg to about 500 mg, and aspirin from about 125 mg to about 500 mg per unit dose.

7. The composition of claim 5, wherein paraxanthine is present at about 75 mg, acetaminophen at about 250 mg, and aspirin at about 250 mg per unit dose.

8. The composition of claim 5, wherein the weight ratio of paraxanthine to acetaminophen to aspirin is approximately 1:3-4:3-4.

9. The composition of claim 5, further comprising magnesium in an amount from about 25 mg to about 100 mg per unit dose.

10. The composition of claim 5, further comprising riboflavin (vitamin B2) in an amount from about 10 mg to about 50 mg per unit dose.

11. A method of treating headache pain in a human subject comprising administering to the subject a unit dose of a pharmaceutical composition comprising: (a) paraxanthine (1,7-dimethylxanthine) in an adjuvant-effective amount; and (b) at least one analgesic agent selected from the group consisting of acetaminophen, aspirin, ibuprofen, naproxen, and naproxen sodium;
wherein the paraxanthine enhances the analgesic efficacy of the at least one analgesic agent via adenosine A1 and A2A receptor antagonism.

12. The method of claim 11, wherein the headache pain is selected from the group consisting of tension-type headache, migraine headache with aura, migraine headache without aura, and cluster headache.

13. The method of claim 11, wherein the at least one analgesic agent comprises acetaminophen and aspirin.

14. The method of claim 13, wherein paraxanthine is present at about 75 mg, acetaminophen at about 250 mg, and aspirin at about 250 mg per unit dose, and the composition is free of caffeine.

15. The method of claim 11, wherein the onset of analgesic effect occurs within approximately 30 minutes of administration.

16. A method of enhancing the analgesic efficacy of at least one analgesic agent selected from the group consisting of acetaminophen, aspirin, ibuprofen, naproxen, and naproxen sodium, the method comprising co-administering the at least one analgesic agent with paraxanthine (1,7-dimethylxanthine) in a unit dose, wherein: (a) paraxanthine competitively antagonizes adenosine A1 and A2A receptors; (b) the adenosine receptor antagonism produces cerebral vasoconstriction and potentiates the analgesic effects of the at least one analgesic agent; wherein the co-administration of paraxanthine increases the analgesic efficacy of the at least one analgesic agent by at least approximately 30% compared to the analgesic agent administered alone.

17. The method of claim 16, wherein the analgesic potentiation is at least approximately 40%.

18. The method of claim 16, wherein paraxanthine is present in an amount from about 25 mg to about 150 mg per unit dose.

19. A method of treating headache pain in a human subject with reduced xanthine-related side effects compared to a caffeine-containing analgesic composition, the method comprising administering to the subject a pharmaceutical composition comprising: (a) paraxanthine (1,7-dimethylxanthine) in an adjuvant-effective amount; (b) at least one analgesic agent selected from the group consisting of acetaminophen, aspirin, ibuprofen, naproxen, and naproxen sodium; wherein the composition is free of caffeine; and wherein administration produces analgesic efficacy comparable to or exceeding that of a composition comprising the same analgesic agent(s) with caffeine, while producing reduced incidence of at least one side effect selected from jitteriness, anxiety, nervousness, insomnia, and cardiovascular stimulation.

20. The method of claim 19, wherein the reduced side effects comprise reduced jitteriness and reduced anxiety compared to administration of an equivalent composition containing caffeine in place of paraxanthine.

21. The method of claim 19, wherein the at least one analgesic agent comprises acetaminophen and aspirin, and paraxanthine is present at about 75 mg, acetaminophen at about 250 mg, and aspirin at about 250 mg per unit dose.

22. The composition of claim 5, wherein the composition is formulated as a tablet or caplet for oral administration.

23. The composition of claim 5, wherein the composition is formulated as a hard gelatin or hydroxypropyl methylcellulose (HPMC) capsule.

24. The composition of claim 5, wherein the composition is formulated as a powder packet for dissolution in water.

25. The composition of claim 5, wherein the composition is formulated as an effervescent tablet comprising sodium bicarbonate and citric acid for dissolution in water.

26. The composition of claim 5, wherein the composition is formulated as an orally dissolving tablet (ODT) for administration without water.

27. A pharmaceutical composition for the treatment of migraine headache comprising, in a unit dose: (a) paraxanthine (1,7-dimethylxanthine) in an amount from about 25 mg to about 150 mg as an adenosine A1 and A2A receptor antagonist; (b) acetaminophen in an amount from about 125 mg to about 500 mg; and (c) aspirin in an amount from about 125 mg to about 500 mg; wherein the composition is free of caffeine; and wherein the composition provides migraine pain relief within approximately 30 minutes of oral administration.

28. The composition of claim 27, further comprising ginger extract (Zingiber officinale) in an amount from about 25 mg to about 75 mg as an anti-nausea agent for migraine-associated nausea.

29. The composition of claim 27, wherein paraxanthine is present at about 75 mg, acetaminophen at about 250 mg, and aspirin at about 250 mg.

30. A kit for headache pain relief comprising: (a) a plurality of unit dose packets or blister packs, each comprising a pharmaceutical composition containing paraxanthine (1,7-dimethylxanthine) in an adjuvant-effective amount and at least one analgesic agent selected from the group consisting of acetaminophen, aspirin, ibuprofen, naproxen, and naproxen sodium; and (b) instructions directing the user to administer one unit dose at onset of headache pain, with optional re-dosing as appropriate for the specific analgesic agent, not to exceed the recommended daily maximum for each analgesic agent.

31. A pharmaceutical composition for the treatment of pain comprising, in a unit dose: (a) paraxanthine (1,7-dimethylxanthine) in an adjuvant-effective amount as an adenosine A1 and A2A receptor antagonist for cerebral vasoconstriction and analgesic potentiation; and (b) ibuprofen in an analgesic-effective amount for peripheral COX-1 and COX-2 inhibition; wherein the composition is free of caffeine; and wherein the paraxanthine enhances the analgesic efficacy of ibuprofen by at least approximately 30% via a mechanism independent of COX inhibition.

32. The composition of claim 31, wherein paraxanthine is present in an amount from about 25 mg to about 150 mg and ibuprofen is present in an amount from about 100 mg to about 400 mg per unit dose.

33. The composition of claim 31, wherein paraxanthine is present at about 75 mg and ibuprofen is present at about 200 mg per unit dose.

34. The composition of claim 31, further comprising acetaminophen in an analgesic-effective amount from about 125 mg to about 500 mg, wherein the composition provides three mechanistically independent analgesic pathways: central pain threshold elevation (acetaminophen), peripheral COX inhibition (ibuprofen), and adenosine receptor antagonism (paraxanthine).

35. The composition of claim 34, wherein paraxanthine is present at about 75 mg, ibuprofen at about 125 mg, and acetaminophen at about 250 mg per unit dose.

36. A method of treating headache or pain in a human subject comprising administering to the subject a unit dose of a pharmaceutical composition comprising: (a) paraxanthine (1,7-dimethylxanthine) in an adjuvant-effective amount as an adenosine A1 and A2A receptor antagonist; and (b) ibuprofen in an analgesic-effective amount; wherein the composition is free of caffeine; and wherein the paraxanthine enhances the analgesic efficacy of ibuprofen via cerebral vasoconstriction and analgesic pathway potentiation independent of COX inhibition, and the onset of analgesic effect occurs within approximately 30 minutes of administration.

37. A pharmaceutical composition for the treatment of pain comprising, in a unit dose: (a) paraxanthine (1,7-dimethylxanthine) in an adjuvant-effective amount as an adenosine A1 and A2A receptor antagonist for cerebral vasoconstriction and analgesic potentiation; and (b) naproxen sodium in an analgesic-effective amount for long-duration peripheral COX-1 and COX-2 inhibition; wherein the composition is free of caffeine; and wherein the paraxanthine enhances the analgesic efficacy of naproxen sodium by at least approximately 30%, and the combination provides rapid onset via paraxanthine-mediated vasoconstriction with sustained duration via naproxen sodium's extended COX inhibition.

38. The composition of claim 37, wherein paraxanthine is present in an amount from about 25 mg to about 150 mg and naproxen sodium is present in an amount from about 110 mg to about 440 mg per unit dose.

39. The composition of claim 37, wherein paraxanthine is present at about 75 mg and naproxen sodium is present at about 220 mg per unit dose.

40. A method of treating headache or pain in a human subject comprising administering to the subject a unit dose of a pharmaceutical composition comprising: (a) paraxanthine (1,7-dimethylxanthine) in an adjuvant-effective amount as an adenosine A1 and A2A receptor antagonist; and (b) naproxen sodium in an analgesic-effective amount; wherein the composition is free of caffeine; and wherein the composition provides rapid analgesic onset within approximately

30. minutes via paraxanthine-mediated cerebral vasoconstriction and sustained analgesic duration of at least approximately 8 hours via naproxen sodium's extended COX inhibition.
